(19) United States
(12) Patent Application Publication
(10) Pub. No.: US 2011/0267577 A1
(43) Pub. Date: Nov. 3, 2011

(54) OPHTHALMIC DIAGNOSTIC APPARATUS

(76) Inventor: Dinesh Verma, Warwickshire (GB)

(21) Appl. No.: 13/061,689

(22) PCT Filed: Aug. 19, 2009
(86) PCT No.: PCT/GB09/51034

§ 371 Date: Jul. 15, 2011

(30) Foreign Application Priority Data
Sep. 1, 2008 (GB) 0815859.4

(51) Int. Cl. A61B 3/02; H02J 7/00; A61B 3/06; H05K 5/00; A61B 3/08; A61B 3/032

(52) U.S. Cl. 351/201; 351/239; 351/242; 361/679.01; 351/223; 320/107; 351/246

(57) ABSTRACT

The present invention relates to an ophthalmic diagnostic apparatus. The apparatus comprises: a display means (12) arranged to be worn in proximity to a user's eyes and arranged for displaying images relating to at least one visual function test to a user; a first user interface (14) for providing instructions to a user relating to said at least one visual function test and a second user interface (16) for receiving responses from said user in relation to said at least one visual function test; storage means (22) for storing data relating to said at least one visual function test; control means (18) for transferring said data relating to said at least one visual function test to said display means and said user interface; and output means for outputting a signal responsive to the user responses so as to assist with ophthalmic diagnosis.

The present invention relates to an ophthalmic diagnostic apparatus and related method and system and particularly, but not exclusively, to a personal ophthalmic diagnostic apparatus, method and related system.

There is an impending epidemic of ageing in the world. According to latest predictions by the United Nations the number of people over 60 years of age will triple by 2050 reaching almost two billion (Population Challenges & Development Goals. Part 1—World Demographic Trends. Chapter III: Population Ageing, page 13—http://www.un.org/esa/population/unpop.ht Population of over 80s will soar five fold to 379 million.

Age-related macular degeneration (AMD) is currently the most common cause of blindness in the western world already affecting 20-25 million people globally, a number that will triple in 30 to 40 years (Global trends in magnitude of visual impairment—http://www.who.int/mediacentre/factsheets/fs282/en/index.html). By the age of 75 years, 20% of population will suffer from AMD.

The age-standardised cumulative one year incidence of sight threatening AMD in at least one eye is 7760 cases per million (Lacour M, Kiilgaard J. F., Nissen M. H. Drugs and Ageing. Volume 19, Number 2, 1 Feb. 2002, pp. 101-133(33)). In, for example, the United States, with a population of 300 million, this translates to 2.3 million new cases per year, and with all these patients at a risk of developing sight-threatening AMD in the other eye. Furthermore the immediate relatives of these patients and smokers over 50 years of age are also at a much higher risk of developing AMD than the rest of the population. Early diagnosis of this condition is considered key to successful treatment so as to prevent blindness.

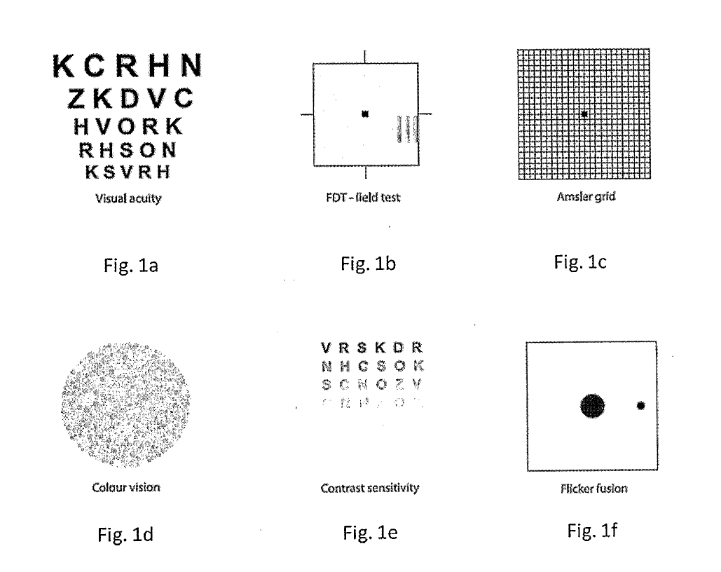

The ability to see or “vision” has many components, which can be affected independent of each other in various eye disorders and are measurable separately. The easiest component to understand is visual acuity, which involves the ability to see very small details using the central and most sensitive part of the retina. Other functions comprise the ability to differentiate colours (colour vision), the ability to discern laterally spaced features without moving the eyes (peripheral or field of vision), the ability to differentiate changing darkness or lightness of letters or objects (contrast sensitivity) and the ability to appreciate distances of objects from us (depth perception or stereopsis). Tests have been devised to measure such visual functions separately, and examples of test patterns employed in such tests are illustrated in FIGS. 1a to 1f.

As stated above, an eye problem that is becoming increasingly common with the ageing population is AMD, In the more serious form of this condition (wet-AMD) abnormal vessels develop under the retina leaking fluid or blood and causing disturbance of the normally smooth layer of retinal cells which detect light (photoreceptors). A patient will typically present with distortion of vision and to prevent the condition becoming serious, fairly urgent treatment is required. If a patient already has this condition in its less serious form (dry-AMD), or is at high risk of developing this condition, for example if there is a family history of AMD, or they are a smoker, have high blood pressure or suffer from obesity, it is necessary to monitor their central sharp vision to readily identify such distortion. A test that can detect very early ageing changes in the retina is known as flicker fusion and employs an image such as that of FIG. 1f.

Colour vision can be affected by various genetic disorders but the problem is becoming more common because many pharmaceutical drugs used for chronic conditions like arthritis and impotence can also affect it. Patients taking these medicines need to monitor their colour vision to detect the early emergence of such side effects.

Glaucoma is another relatively common condition and affects in the order of 2% (5% of over 75 years old) of the population. This is a chronic condition that affects the field of vision and is usually treated with long-term medication. Patients for this condition require regular monitoring of their visual fields by way of a test pattern such as that of FIG. 1b.

Other displayed images are available for testing and monitoring visual functions in general. The simplest is a chart with white background and black letters of varying sizes known as Snellen's chart (see FIG. 1a) available at most primary care physicians and opticians. This chart is intended to be held at 6 metres (20 feet) away from the reader and the visual acuity (function of vision by which we see smallest of objects) is measured depending on the smallest letters that can be read in comparison to general population norms. So if a patient can read the letters in a line that a person having normal vision can read at 6 metres (20 feet) then the visual acuity is recorded as “6/6” (or “20/20”). In the other extreme, if a person can only read for example the larger letters in the top row at 6 metres (20 feet) which a normal sighted person could read from 60 metres (200 feet) then that person's visual acuity is recorded as “6/60” (or “20/200”). Though this test has been very popular for many years, there are various problems with its reliability because there is no control on many variables such as illumination of the chart, a patient's ability to guess or remember letters, variation of distance from where it is read, possibility of wilful or inadvertent cheating when closing one eye, etc. Due to these reasons, various modifications of Snellen's chart have been introduced in recent years, which control many of the variables by using projectors or computer screens. While these can increase the test-reliability, the associated cost of the device becomes much higher (approximately £2,500 per device).

A current method of monitoring distortion of vision is known as Amsler's test and employs the image of a grid (small squares within a large square) with a central spot printed on paper or card (see FIG. 1c). A patient is asked to cover one eye and look at the central spot keeping the paper at around 33 cm from the face. For a person of normal sight all the squares look regular and the same size but if there is any distortion in central vision then the squares may appear to have different sizes. Also some squares may appear to be missing or blind spots may appear in the grid. It is intended that the patient draw on these areas of distortion and blind spots to monitor the progress of their condition. This test has very obvious drawbacks due to lack of standardisation and difficulty of self-administration of the test. A new computer based device called a Hyperacuity meter has entered the market and which improves the reliability of the “distortion testing”, but the cost of the device is rather high (approximately £5000) and also requires a professional to deliver the test.

Other visual functions such as field of vision (a measure of how much can be seen around in the periphery of vision without moving the eyes) are tested and monitored using more complex computerised, single function devices, which are very expensive (approximately £25000).

Colour vision is currently tested using a standardised book that has various numbers or letters made up of particular colour dots embedded amongst dots of similar colours which are difficult to differentiate for a person who has deficiency of colour vision. This is called an “Ishihara test” after its inventor Dr. Ishihara (see FIG. 1d). Even though this particular test is not very expensive, due to the variability of viewing conditions, it may also miss early colour vision defects and hence new computer based tests are being introduced.

Contrast sensitivity is another subtle visual function that is affected very early in many common eye disorders like cataracts. This test measures the ability of the eye to differentiate letters of varying contrasts (see FIG. 1e).

Depth perception, or stereopsis, is tested using slides or plates, which allow a person to see slightly disparate images from each eye, and the ability to fuse that image to give a three-dimensional perception is then measured. Another method is to ask the patient to remove parallax in vertical lines projected at different distances and measuring the distance required to move the image to align the lines. This method is used to regularly monitor stereopsis in pilots and navigators of all commercial airlines.

Traditional tests to measure various visual functions are not very reliable due to uncontrollable variables and lack of measurable standardization. Computer based testing of visual functions is becoming common in recent years with advances in technology to improve the reliability, repeatability and standardisation of these tests.

This has resulted in development of very expensive and complex computer based equipment for testing various visual functions. However, separate apparatus are required for testing each individual visual function separately. This equipment also requires a professional to administer the test and record and interpret the results, increasing the real cost of screening/monitoring much more.

In the particular case of AMD, currently available diagnostic devices for detecting AMD are very expensive, stand-alone units requiring an expert technician to perform the tests. The interpretation of the results is complex and only professionals (optometrists or ophthalmologists) can give an opinion on the significance. Instance access to these professionals can be very difficult and is costly, resulting in inevitable delay in diagnosis affecting ultimate visual potential. Due to the costs involved, the eye care professionals usually perform only a six monthly screening.

The present invention seeks to provide for an ophthalmic diagnostic apparatus having advantages over known such ophthalmic diagnostic apparatus.

The present invention attempts to address the above problems by providing for an ophthalmic diagnostic apparatus and system which can provide basic screening and which can be used by patients themselves at much more frequent intervals than they would otherwise see a professional to monitor their eye functions.

According to one aspect of the present invention there is provided an ophthalmic diagnostic method including the steps of presenting an image comprising a circle having a thickness determined by the results of a Landolt C test, and determining if any distortion/movement of the circle is perceived.

Preferably, the thickness of the circle can correspond to the minimum thickness perceived by way of the Landolt C test.

The method can further include the step of increasing the diameter of the circle as presented in the said image.

Yet further it can advantageously include the step of increasing the diameter of the circle so as to cover substantially the whole of the central macula.

Preferably, the method includes the permission of a central target within the image of the circle.

In particular the said the target can comprise a red target element.

The said image comprising the circle can preferably be presented for a predetermined period, which can be in the region of three seconds.

Yet further, the method can include the step of presenting the image comprising the circle alternately to each eye.

According to another aspect of the present invention there is provided an ophthalmic diagnostic apparatus including means for presenting an image comprising a circle having a thickness determined by the result of a Landolt C test, so as to allow for the user of the apparatus to determine any perceived distortion/movement to the image of the circle.

Preferably the apparatus is arranged for use in accordance with the step of a method as outlined above.

Further, the said ophthalmic diagnostic apparatus can comprise: display means arranged to be worn in proximity to a user's eyes and arranged for displaying images relating to at least one visual function test to a user; a first user interface for providing instructions to a user relating to said at least one visual function test and a second user interface for receiving responses from said user in relation to said at least one visual function test; storage means for storing data relating to said at least one visual function test; control means for transferring said data relating to said at least one visual function test to said display means and said user interface; and output means for outputting a signal responsive to the user responses so as to assist with ophthalmic diagnosis.

The invention is advantageous in that it enables people to monitor their vision on a more regular basis and at a time and place more suitable to their requirements. Further, it will aid better understanding of the eye condition by a patient and support the work of both community opticians as well as hospital ophthalmologists in their monitoring of progression of the condition. The need to make unnecessary trips for routine check ups will be reduced if the patient is reassured by the invention that their sight is stable. It will also allow earlier detection of disease progression and therefore early treatment which usually has a better outcome and prognosis than if left until an annual check-up. Also, the invention is multifunctional and can provide a number of visual function tests on the same device.

The apparatus can further comprise a processor for processing data relating to user responses to the at least one visual function test received via the second user interface and for forwarding processed data to said output means for output as said signal responsive to the user responses.

Conveniently, said output means includes transmission means for transmitting said signal responsive to the user responses over a communication network.

Further, said signal responsive to the user responses is transmitted to a remote location.

In particular, said communication network can include an intermediate transceiver element between said transmission means and said remote location, and wherein, upon reception of said signal responsive to the user responses, said intermediate transceiver is arranged to forward signal responsive to the user responses to said remote location.

Also, transmission of said signal responsive to the user responses over said communication network can comprise wireless transmission.

If required, said control means further comprises a receiving means for receiving data transmitted over said communication network from a remote location.

Preferably, said output means is arranged to output said signal responsive to the user responses directly to said user.

Conveniently, said control means is further arranged to forward data relating to said signal responsive to the user responses for storage in said storage means.

Further, said first user interface comprises visual means incorporated in said display means.

In particular, the apparatus is arranged such that user instructions are presented to a user through said visual means.

Also, said display means is arranged to display three-dimensional images.

If required, said three-dimensional images are formed from a pair of two-dimensional images.

Preferably, a first two-dimensional image of said pair of two-dimensional images can be displayed on a first screen of said display means and a second two-dimensional image of said pair of two-dimensional images is displayed on a second screen of said display means, where said first screen is arranged for location in proximity to one eye of the user and a second screen is arranged for location in proximity to the other eye of the user.

Conveniently, said first image and said second image represent two perspectives of the same image to mimic the perspectives that both eyes of a user naturally receive in binocular vision.

Further, said first user interface can comprise an audio signal transducer.

In particular, said second user interface comprises the, or a further, audio signal transducer.

Also, said apparatus is arranged such that user instructions are presented to a user through an earpiece.

If required said second user interface includes a microphone.

Preferably, said user responses are audio responses recorded via said microphone.

Conveniently, said control means is configured to operate a voice recognition process for capturing and processing said audio responses of a user.

Further said second user interface includes tactile elements.

In particular, said tactile elements comprise user operable switches.

Also, said apparatus is arranged such that said at least one visual function test comprises a visual acuity test.

If required, said apparatus is arranged such that said at least one visual function test comprises a test for glaucoma.

Preferably, said apparatus is arranged such that said at least one visual function test comprises a test for monitoring distortion of vision.

Conveniently, said apparatus is arranged such that said at least one visual function test comprises a colour vision test.

Further, said apparatus is arranged such that said at least one visual function test comprises a test for monitoring contrast sensitivity.

In particular, said apparatus is arranged such that said at least one visual function test comprises a test for detecting changes in a retina.

Also, said apparatus further can comprise an integral power source.

If required, said integral power source can comprise a rechargeable battery.

Preferably, said apparatus can be arranged in a headset.

Conveniently, said apparatus can comprise goggles.

Further, said display means is located in a lens portion of said goggles.

In particular, said first user interface is located in a lens portion of said goggles.

Also, said first user interface is located in an earpiece portion of said goggles.

If required, said second user interface is arranged for location in proximity to a mouth of a user.

Preferably, said headset comprises spectacles.

Further, said display means comprises a liquid crystal display.

Alternatively, said display means comprises an organic light emitting diode display.

According to another aspect of the present invention, there is provided a docking station for receiving the apparatus described above, wherein said docking station comprises a protective case comprising a main housing for receiving the said ophthalmic diagnostic apparatus.

Preferably, said docking station further comprises a lid portion serving to enclose said ophthalmic diagnostic apparatus in said main housing when in a closed position and to allow access to said ophthalmic diagnostic apparatus when in an open position.

Conveniently, said docking station further comprises a power supply cable to provide electrical power to said docking station and a means to electrically couple said ophthalmic diagnostic apparatus to said power supply when located within said docking station so as to recharge said rechargeable battery.

Further, said intermediate transceiver element is located within said docking station.

According to a further aspect of the present invention, there is provided an ophthalmic diagnostic system comprising: an ophthalmic diagnostic apparatus as described above; a communication network; and a remote server, wherein a signal responsive to the user responses entered via said second user interface is transmitted from said ophthalmic diagnostic apparatus to said remote server via said communication network.

Preferably, data relating to corrective action proposed by an ophthalmic specialist based upon data received via said signal responsive to the user responses is transmitted from said remote server to said ophthalmic diagnostic apparatus via said communication network.

Conveniently, the ophthalmic diagnostic system described above further comprises the docking station described above.

It should of course be appreciated that the present invention can also comprise a computer program product including instructions which, when loaded on a computer, serve to control a method as outlined above.

The present invention is described further hereinafter, by way of example only, with reference to the accompanying drawings in which:

FIG. 1a illustrates a conventional Snellen chart used in the testing of visual acuity;

FIG. 1b illustrates a conventional FDT field test used in the testing of glaucoma;

FIG. 1c illustrates a conventional Amsler test used to monitor distortion of vision;

FIG. 1d illustrates a conventional Ishihara test used in the testing of colour vision;

FIG. 1e illustrates a conventional test for monitoring contrast sensitivity in the vision of a patient;

FIG. 1f illustrates a conventional flicker fusion test for detecting changes in the retina of a patient;

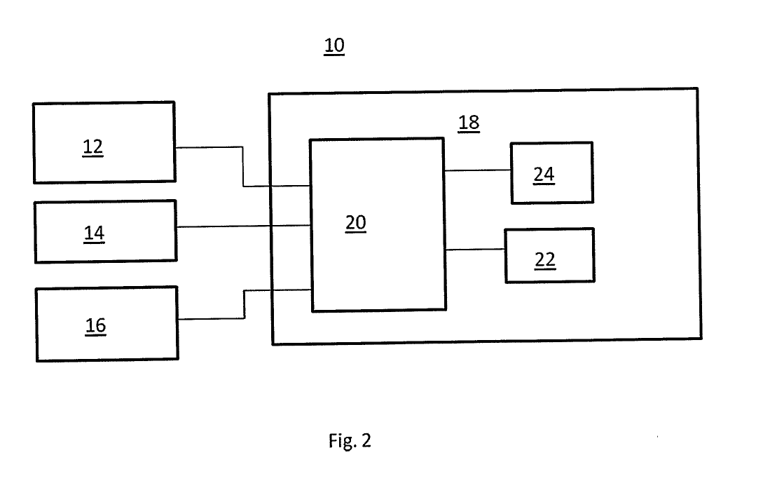

FIG. 2 illustrates a schematic diagram of a personal ophthalmic diagnostic apparatus according to an embodiment the present invention;

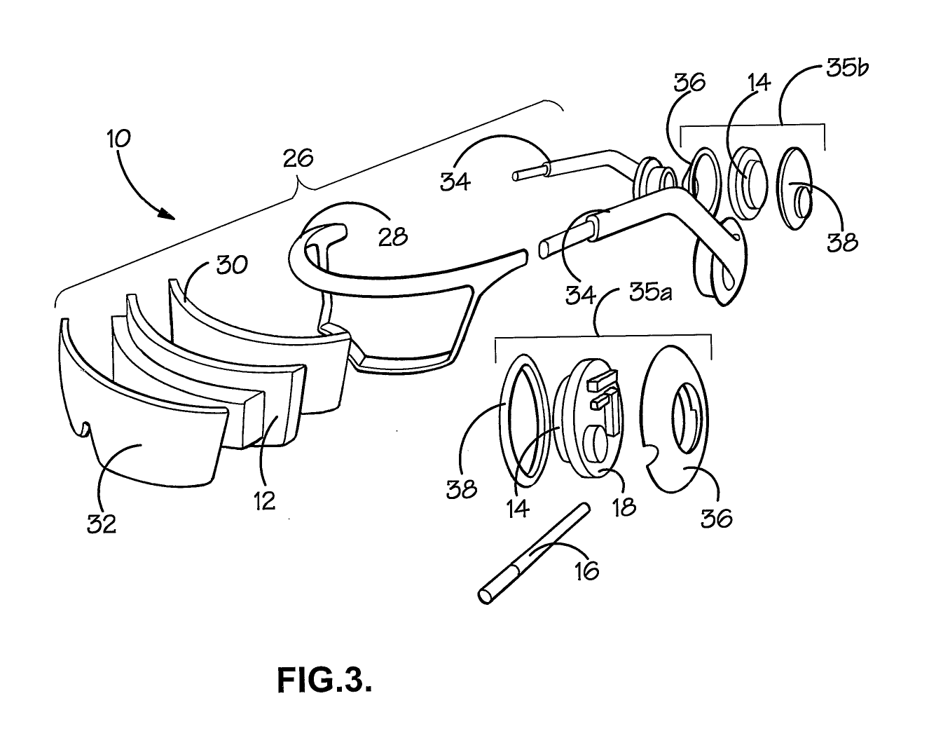

FIG. 3 illustrates an exploded perspective view of the personal ophthalmic diagnostic apparatus according to the present invention;

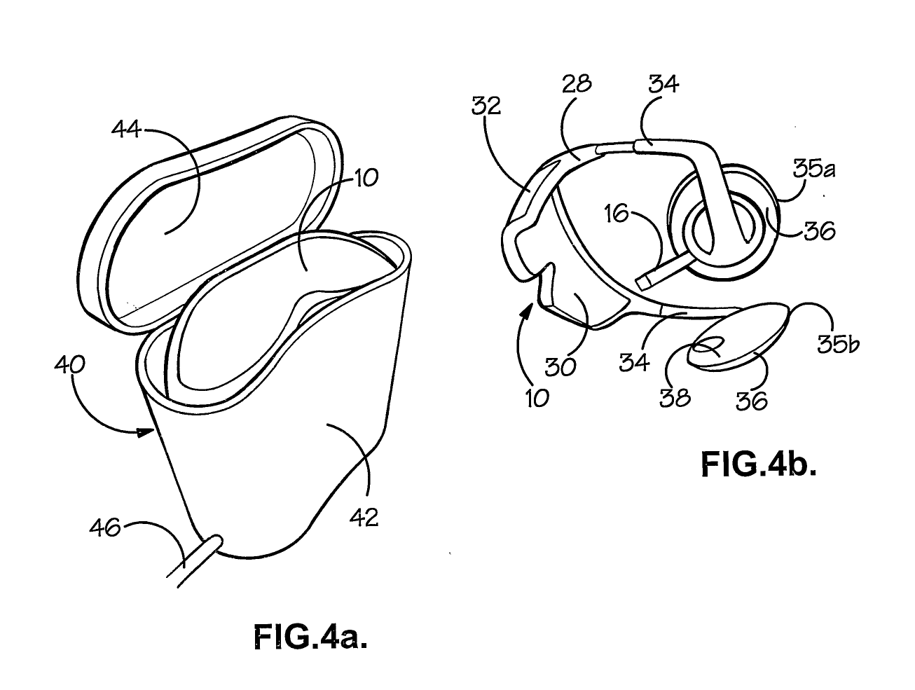

FIG. 4a illustrates a protective case for said personal ophthalmic diagnostic apparatus with said personal ophthalmic diagnostic apparatus located therein;

FIG. 4b illustrates a perspective view at a different angle to that of FIG. 3 of the personal ophthalmic diagnostic apparatus according to the present invention;

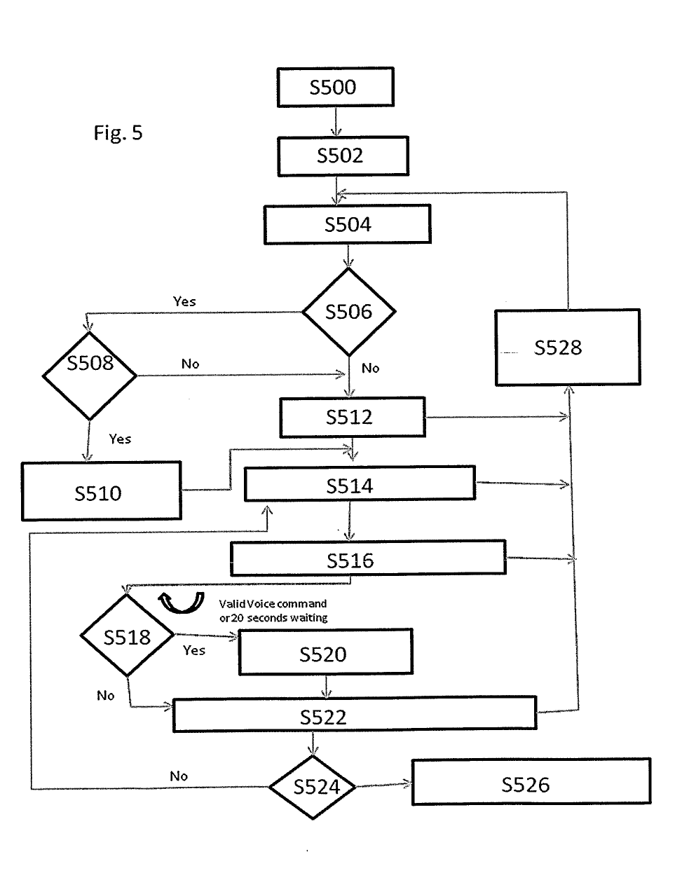

FIG. 5 illustrates the steps which can be performed during a visual function test;

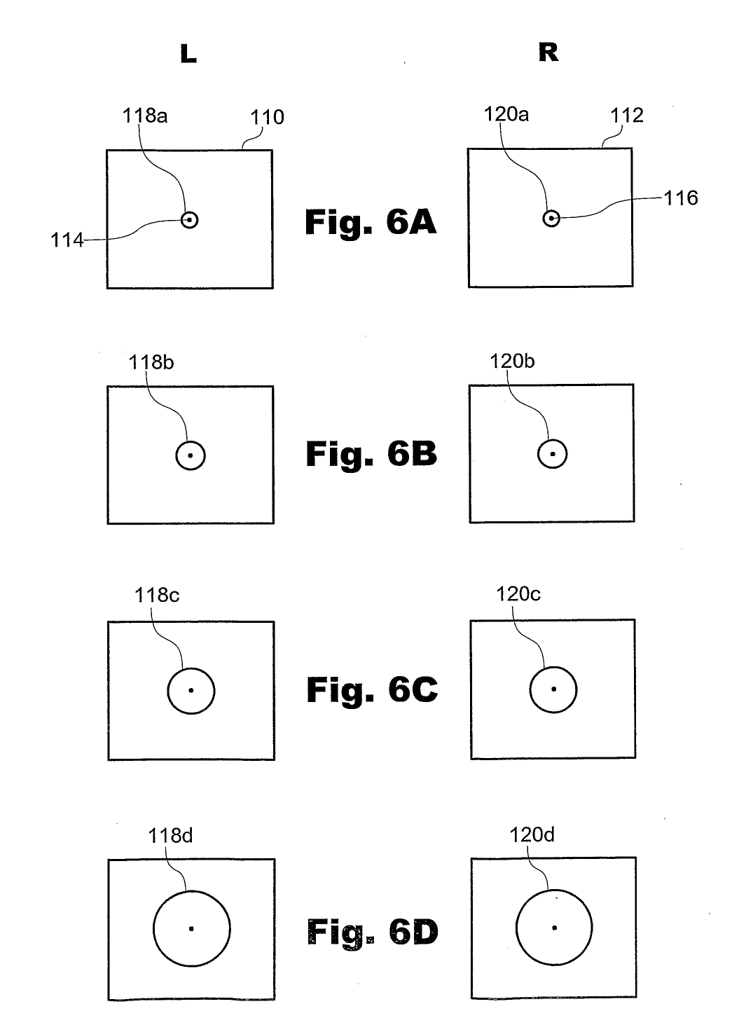

FIGS. 6a-6d illustrate image displays according to a method and apparatus of one particular embodiment of the present invention; and

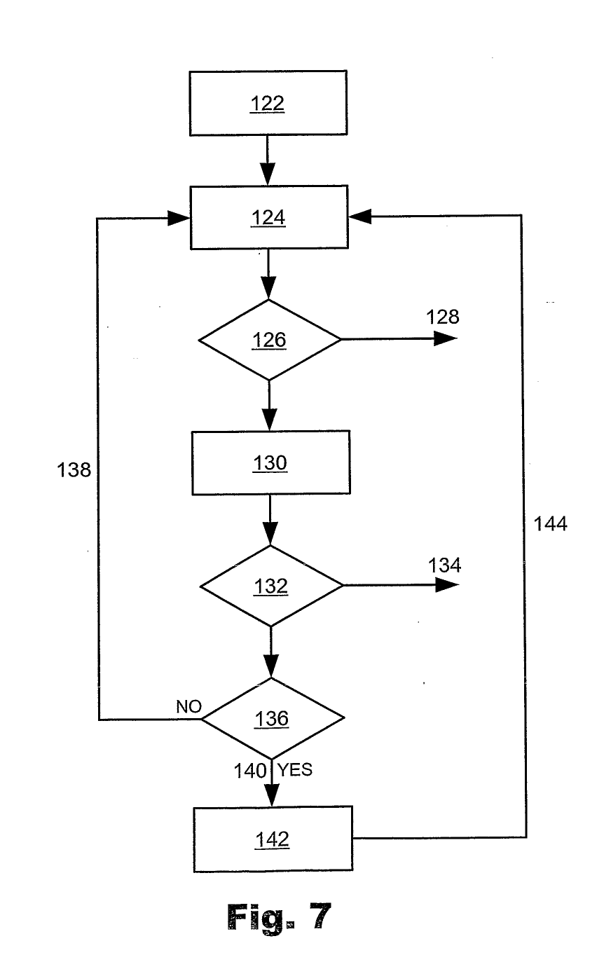

FIG. 7 is a flow diagram illustrating one embodiment of the operational system offered by the present invention.

FIG. 2 illustrates the personal ophthalmic diagnostic (POD) apparatus 10 in schematic form. The POD apparatus 10 comprises a display means 12, a loudspeaker 14, a user interface 16, and a control means 18. The control means 18 comprises a processor 20, a memory 22 and a transmitter 24.

Each of the display means 12, loudspeaker 14, user interface 16, memory 22 and transmitter 24 are electronically coupled to said processor 20 to enable data transfer between the processor 20 and these elements, and vice versa.

The POD apparatus 10 is arranged to allow an unskilled user to conduct at least one visual function test without requiring a skilled professional. Obviously an unskilled user will not be able to take the appropriate corrective action if, after conducting the at least one visual function test, visual defects are found, but rather the POD apparatus 10 is arranged to provide an early warning regarding possible visual defects and is arranged to advise the user to seek professional advice and/or the POD apparatus 10 is arranged to transmit the results of the at least one visual function test conducted by the user to a remote server for review by a professional.

The POD apparatus 10 is arranged to display these at least one visual function tests on the display means 12. Instructions regarding the at least one visual functions test are transmitted to the user by means of loudspeaker 14, and/or display means 12, and the responses of the user are received via user interface 16 (e.g. a microphone). The received responses are then forwarded to the processor 20 which processes the responses and then forwards the results for storage in memory 22.

In addition to storing the results of the at least one visual function test in memory 22, the processor also forwards the results to said transmitter 24 for onward transmission to, for example, a remote server. The results are then stored at the remote server for review either immediately, or at a later time, by an ophthalmic specialist. Based upon the results reviewed by the specialist, the specialist can then contact the patient, if necessary, to arrange for corrective action to be taken.

As an alternative, or in addition to the above, the processor 20, after processing of the user responses to create test results, may automatically compare the current test results with previously recorded test results (which may be stored in memory 22) to determine if there is any discrepancy or deterioration in visual functions. Indeed, if discrepancies or deterioration in visual functions are discovered the specialist can, as described above, contact the patient to arrange for corrective action to be taken.

The POD apparatus 10 of the present invention is arranged to be compatible with a communication network such that data relating to visual function test results can be transferred to a remote server as described above. In this regard, the transmitter 24 of the POD apparatus 10 may comprise mobile phone technology for transmitting the test results to the server. Alternatively, or additionally, the transmitter 24 of the POD apparatus 10 may comprise WiFi technology for transmitting said test results to a network port coupled to the communication network. In an alternative to this arrangement, the test results may be transmitted from the POD apparatus 10 to the network port via a cable. Preferably, the network port is coupled to the communication network by way of a cable, but may be coupled to the communication network by any suitable means (wired or wireless).

As will be appreciated, the processor 20 of the apparatus 10 in FIG. 2 can advantageously be arranged to present an image comprising a circle having a thickness determined by a result of a Landolt C test to which a patient can either independently previously have been subjected, or which can be provided by way of the same diagnostic session by way of the apparatus 10 and processor 20 and related disclaiming 12. As will be described later with reference to FIGS. 6a-6d the processor 20 can be arranged to display, inventive step wireless fashion, and for a series of circles of ever increasing diameter so as programs cover the whole central macular of the user/patient.

For each diameter, the image is advantageously presented or in the order of three seconds and alternatively provided for each eye for the region of six times per eye.

By way of the user interface 16, the user/patient can readily record his/her perception of the circles of increasing diameter recording, in particular, any such perceived distortion/movement of the circle.

The patient can advantageously be prompted to provide a response each time the image is presented by way of the disclaiming 12, if a central fixation target in employed, the user/patient can likewise be reminded to keep his/her focus up on that central fixation target at all times.

At the end of the image-display session, a feedback result can be provided to the user by way of the interface and/or the loudspeaker 14 and/or user interace 16, which results will be dependent upon the audio responses previously provided by the user/patient and may, for example, provide a prompt for the user/patient to refer to an eye care specialist perhaps on an urgent basis.

FIG. 3 illustrates an exemplary arrangement of the POD apparatus 10 in a preferred embodiment of the present invention. The features illustrated in FIG. 3 which correspond to features already described in relation to FIG. 2 are denoted by like reference numerals.

In the preferred embodiment as illustrated in FIG. 3, the POD apparatus 10 is realised in a pair of spectacles/goggles 26, but referred to hereinafter as spectacles. The spectacles 26 comprise a frame 28 arranged to receive an inner lens 30 and an outer lens 32 which, in turn, are arranged to receive therebetween display means 12.

Substantially parallel arms 34 extend from the frame 28 and have located at the remote ends thereof earphone arrangements 35a, 35b.

When in use, the spectacles 26 are worn such that the frame 28 is located adjacent the eyes of the user (thus, the user can view display means 12 through inner lens 30) with the arms 34 extending around the sides of the user's head such that the earphone arrangements 35a, 35b are located over the user's ears.

In the illustrated embodiment, earphone arrangement 35a comprises an earphone casing 36 which houses said control means 18 and said loudspeaker 14. Earphone arrangement 35b is similar (i.e. comprises an earphone casing 36 and a loudspeaker 14), but does not possess a control means. In alternative arrangements, the control means 18 may be located in one earphone arrangement 35a or 35b, and a loudspeaker 14 may be located in the other earphone arrangement 35b or 35a, or the control means 18 may be located in both earphone arrangements 35a, 35b and the loudspeaker may be located in only one of the earphone arrangements 35a, 35b.

Preferably the earphone arrangements 35a, 35b include earphone cushions 38 to minimise discomfort to a user caused by pressure of the earphone arrangements 35a, 35b upon the user's ears when the POD apparatus 10 is in use.

As illustrated in FIG. 3, user interface 16 (microphone) is located in an arm extending from earphone arrangement 35a and is arranged such that, when the POD apparatus 10 is in use, the user interface 16 can receive voice input from said user.

Preferably, the spectacles 26 of the POD apparatus 10 are based upon a spectacle-based platform similar to virtual reality spectacles for watching DVD films or playing 3-D video games. The display means 12 preferably comprises two separate LCD (or organic light emitting diode (OLED)) screens, i.e. one for each eye.

Conveniently, the display means 12 implements stereoscopy, stereoscopic imaging or 3-D (three-dimensional) imaging, i.e. any technique capable of presenting the illusion of depth in an image. This is done by presenting a slightly different image to each eye and so a 3-D image is created from a pair of 2-D images. Depth perception in the brain is provided by presenting the user with two different images representing two perspectives of the same object, with a minor deviation similar to the perspectives that both eyes naturally receive in binocular vision.

Such techniques will be apparent to those skilled in the art.

In addition to the screens for displaying images to said user, said display means 12 also comprise the electronic devices required to drive the screens.

When a user conducts a visual function test using the POD apparatus 10, the images for each visual function test are projected/displayed on the display means 12 to appear as though viewed from the standardised distances of 33 cm or 6 metres (20 feet) depending upon the test. Instructions are transmitted through the loudspeakers 14 in the earphone arrangements 35a, 35b and the responses of the user are received by the user interface 16 which transfers the received voice data to the processor 20.

In the present embodiment, the processor 20 is configured to act upon the voice data by means of voice recognition software, The voice data is transformed to data representing the results of the user responses, and is subsequently stored in memory 22. In addition, the processor 20 compares the results with previously recorded results to determine if there is any discrepancy or deterioration in visual functions. If this is indeed the case, the processor 20 is arranged to forward the results of the test via transmitter 24 to a remote server, where the results are stored pending review by an eye care professional.

The POD apparatus 10 of the present invention allows a user to conduct visual function tests (such as those described in relation to FIGS. 1a to 1f) without requiring separate testing devices. It should be appreciated that the actual visual function tests provided by the POD apparatus 10 of the present invention may differ from those illustrated in FIGS. 1a to 1f (since those tests are merely examples of conventional tests). Indeed, the visual function tests provided by the POD apparatus are not limited to those as illustrated in FIGS. 1a to 1f.

In an alternative to the above described embodiment, transmitter 24 may be replaced with a transceiver. The transceiver would function in the same way as the transmitter 24 described above, but would also allow the POD apparatus 10 to receive signals. Such an arrangement would allow a user in possession of the POD apparatus 10 to receive reminders that a test with an eye care professional is required. The POD apparatus 10 may therefore usefully be provided with an alarm which sounds and/or an LED which flashes when a reminder is received that an eye test is due.

In addition to the arrangement above whereby the test results are transmitted to an eye care professional, the POD apparatus 10 may also incorporate a system which provides user feedback regarding the test results, i.e. once the test results are interpreted they are reported back to the user through loudspeakers 14.

As will be appreciate by the person skilled in the art, the spectacles 26 of the POD apparatus 10 described above include a power source, but this power source has not been described/illustrated for reasons of clarity. Preferably, however, the power source comprises a rechargeable battery.

A protective case 40 is illustrated in FIG. 4a. This protective case 40 is arranged to retain and protect the POD apparatus 10, for example, during periods of non-use. The protective case 40 comprises a main compartment 42 arranged to receive and house the POD apparatus 10, and a lid portion 44 which is hingedly attached to the main compartment 42. The lid portion 44 serves to enclose the POD apparatus 10 in the protective case 40 when in the closed position, and allow access to the POD apparatus 10 when in the open position.

The protective case 40 also comprises a power supply cable 46 to provide electrical power to the protective case 40. In this regard, the protective case 40 acts as an adapter, to which the POD apparatus 10 can be electrically coupled, to allow charging of the power source of the POD apparatus 10. When the POD apparatus 10 is located in the protective case 40, an indicator located on the protective case 40 may indicate if the POD apparatus 10 requires charging or has completely charged (e.g. a red light to indicate lack of charge and a green light to indicate full charge).

Preferably, the protective case 40 is linked to a communication network so that, when the POD apparatus 10 is located within the protective case 40, data stored in the POD apparatus 10 can be transferred to the protective case 40 for onward transmission, via the communication network, to a remote server. The network port referred to earlier could conveniently be located in said protective case 40.

Although the lid portion 44 is attached to the main compartment 42 by a hinge in the above embodiment, it may be attached by any suitable means.

FIG. 4b merely illustrates the POD apparatus 10 in unexploded form and from a different angle to that of FIG. 3. However, apart from these differences, the illustrated features are the same, and so are denoted by like reference numerals and will not be discussed further.

In another embodiment of the present invention, a POD system for allowing a user to conduct visual function tests can reside as an application on a mobile phone. The mobile phone is connected to the spectacles 26 described above by way of a cable (or alternatively wirelessly by, for example, Bluetooth).

A menu tree displaying a number of menu options may be displayed on a screen of the mobile phone in the above described preferable embodiment. The menu tree will offer the user a choice from the following menu options:
(a) Launch test—This is selected by the user to start the visual function test(s);
(b) Settings—This enables the user to change the settings from the default settings; and
(c) Exit—This enables the user to leave the application.

Upon selection of “Launch test”, the user is taken through the test screens in a step-by-step process, with the user being asked questions relating to each test screen. The test screens may relate to those tests as illustrated in FIGS. 1a to 1f. The user responses are recorded and converted to response data using voice recognition software and the test screen is then updated to display the next test screen. This sequence of displaying a test screen, asking questions and recording the answer will continue until all tests have been conducted.

When the tests have been completed, the results are saved in a simple text format. At this point, the result file can be sent to an eye care professional from the mobile phone either immediately and automatically or later when the user wishes to send it. In the latter case, the user will have to send the result file from where it is stored on the mobile phone.

As described above, current test results may be compared with those of a previous test. However, only if there is deviation in the result from the previous test results, will the report be sent to the eye care professional.

This testing system may include a pause function to allow the user to stop a test at any stage in its progress and then to resume the test from the point at which progress was paused without requiring the user to start again from the beginning.

The “Settings” menu option of the application enables the user to set, control and save the application parameters. The settings which the user may be able to change are:
(a) Volume level for the earpiece;
(b) Speed of the test program;
(c) Location in which the result file will be stored;
(d) Choice of the mode of communication for the result data transfer. The result of the tests could be sent to computer/laptop or other mobile phones using either emails or SMS;
(e) Recipient's (e.g. eye care professional) mail address/mobile phone number.

Although the above embodiment relates to an application residing on a mobile phone, the application could also reside on a personal desktop computer, laptop computer, personal digital assistant, or any other suitable mobile communication device.

FIG. 5 illustrates examples of the steps which can occur during a visual function test in a particular exemplary embodiment of the present invention and irrespective of the device upon which the test might be hosted.

After the application is launched (S500), speech components are initialised (S502), and the user is offered the choice of beginning a new test or resuming a prior, incomplete, test (S504), e.g. a paused test or prior saved incomplete test. The system determines if there is an incomplete test paused/saved or not (S506) and offers the user the choice of continuing with this incomplete test or starting a new test (S508).

If the user chooses to continue with the paused/saved incomplete test, then the system sets the test counters to the appropriate values (S510) and proceeds to display the images and play the accompanying audio files for the visual function test (S514).

If, however, the user chooses to discard the incomplete test and start a new test, then the system proceeds to start a new test (S512) and displays images and plays the accompanying audio files for the visual function test (S514).

In either case, after step S514 the system proceeds to activate its voice recognition elements to accept the user's verbal responses and activates a timing element (S516).

The timing element is arranged to measure the time elapsed after an instruction has been relayed to the user. The system determines if an answer has been submitted before a predetermined amount of time (e.g. 20 seconds) has elapsed (S518). If it is determined that the time has elapsed and there has been no answer by the user, then the user's answer is recorded as “no answer” (S520). This answer is stored in a memory of said system, and the system counters are incremented (S522). However, if it is determined that an answer has been provided before the time has elapsed, the user's response is recorded, stored in the memory of the system, and the system counters are incremented (S522).

The system then determines if the test(s) is/are complete (S524). If not, the process returns to step S514. However, if all tests are deemed complete, the system displays the results to the user and/or forwards the results to an eye care professional and the process is halted (S526).

As can be seen, the pressing of a pause button (e.g. an “Esc” button on a keyboard) (S528), pauses the application and returns the application to step S504 to await subsequent resumption.

Turning now to FIGS. 6a-6d there is illustrated a series of representations of a display for each of the left and right eye as could be provided, for example, by way of the apparatus described hereinbefore.

Each of the left eye display 10 and the right eye display 12 is arranged to present a respective central fixation target element 14, 16 which, in the illustrated embodiment, can comprise a red dot. As confirmed from FIG. 6a-6d, this central fixation target element remains permantely displayed.

Extending centrally around the central fixation target element 14, 16 in each of the displays 10, 12 is a circle 18 in display 10 and a circle 20 in display 12.

The user/patient to whom the images are presented has previously undergone a Landolt C test for visual acuity and the results of that test have been used to determine the thickness of the each circle 18, 20.

The four images for each display 10, 12 illustrated in FIGS. 6a-6d comprise four steps in a multi-step procedure forming a Relative Alternative Image Neutralisation (RAIN) test. As will be appreciated from reference to FIG. 6a-6d at each stage, the diameter of the circle increases as indicated by the representations 18a, 18b, 18c, 18d, and 20a, 20b, 20c, 20d of FIGS. 6a-6d.

At each stage in the procedure, i.e. at each of the four steps illustrated in FIGS. 6a-6d, the patient is prompted for a response as to whether there is any perceived distortion/movement in the image of the circle 18, 20, i.e. whether or not the image is perceived as a true circle.

The user/patient provides his/her response by way of the user/interface described in before, which responses are recorded. At the end of the diagnostic session, results can be returned to the user which, on the basis of their responses, can determine whether or not the patient/user need seek further specialist advice.

While any appropriate cyclical manner of presentation of the circle images can be provided one particular embodiment of the present invention is now described further with reference to FIG. 7.

Turning therefore to FIG. 7, a Landolt C test is first undertaken at step 1 to 2. Although, in the illustrated embodiment, this step 1 to 2 is illustrated as part of the overall procedure it should be appreciated that the Landolt C test can readily be provided prior to, and quite independently of, the core of the procedure of the present invention.

In any case, returning to the illustrated embodiment of FIG. 7, the result of the Landolt C test conducted at step 122 is employed at step 124 wherein an image of a circle having a thickness determined in accordance with the Landolt C test is presented to the left eye of the user/patient and generally surrounding a central fixation target such as illustrated with reference to FIGS. 6a-6d. The image is presented to the left eye at step 124 for a period of three seconds and the user promptly, at step 126, to indicate whether or not there is a perceived distortion/movement in the image of the circle. The users response is provided and recorded as an input into the apparatus as indicated at 128.

The method continues at step 130 through the display of a similar image to the right eye and again for a period of three seconds. Yet again, the user is prompted at step 132 to provide an input 124 to the apparatus indicating whether or not there is any perceived distortion/movement in the image of the circle.

A determination is then made at step 136 as to whether six separate three second displays have been provided to each eye.

If it is determined that less than six separate readings have been provided, the process returns via 138 to repeat steps 124 and 130.

The provision of the determination step 136 allows for the image to be displayed to each eye repeated a number of times: which means the present example is a total of six times.

Once it is determined at step 136 that the image has been displayed to each eye for a total number of six times, the method continues to step 142 where it is determined whether or not the diameter of the circle is to increased for a further series of measurements.

If it is determined at step 142 that the diameter is to be increased, then the procedure returns via 144 to repeat steps 124, 130 and determinations 126, 132 and 136 but with an image of the circle of slightly larger diameter.

Advantageously, the procedure returns through 144 to allow for an ongoing step wise increase in the diameter of the circle in a manner certain to cover the whole central macular of the user/patient.

In order to assist with the accuracy of the process, the apparatus can readily be provided to prompt the user to keep looking at the central fixation target such, in accordance with the embodiment of FIGS. 6a-6d can comprise a central red dot.

Once it is determined at step 142 in FIG. 7 that the maximum diameter of the displayed circle has been achieved, the diagnostic process concludes and an output can be provided based upon the users responses provided repeatedly at steps 126 and 132 within FIG. 7.

Through the user/patient responses, it can readily determined whether or not referral to an eye care specialist is required on an urgent basis or otherwise.

1. An ophthalmic diagnostic method including the steps of presenting an image comprising a circle having a thickness determined by the results of a Landolt C test, and determining if any distortion/movement of the circle is perceived.

2. A method as claimed in claim 1 wherein the thickness of the circle corresponds to the minimum thickness perceived by way of the Landolt C test.

3. A method as claimed in claim 1, and including the step of increasing the diameter of the circle as presented in the said image.

4. A method as claimed in claim 3, and including the step of increasing the diameter of the circle so as to cover substantially the whole of the central macula.

5. A method as claimed in claim 1, and including the provision of a central target within the image of the circle.

6. A method as claimed in claim 5 where the target includes a red target element.

7. A method as claimed in claim wherein the said image comprising the circle is presented for a predetermined period.

8. A method as claimed in claim 7, wherein the said predetermined period is in the region of three seconds.

9. A method as claimed in claim 1 and including the step of presenting the image comprising the circle alternately to each eye.

10. A method as claimed in claim 9 and including presenting the image six times to each eye.

11. A method as claimed in claim 1, where the image is presented by means of an electronic display screen.

12. An ophthalmic diagnostic apparatus including means for presenting an image comprising a circle having a thickness determined by the result of a Landolt C test, so as to allow for the user of the apparatus to determine any perceived distortion/movement to the image of the circle.

13. Apparatus as claimed in claim 12 and arranged to provide for an increase in the diameter of the said circle so as to allow for a repeat of the determination of a perceived distortion/movement.

14. Apparatus as claimed in claim 13 and arranged to provide for an increase in the diameter of the circle so as to cover the whole of the central macula.

15. Apparatus claimed in claim 12 and arranged to present a central target element within the said circle.

16. Apparatus as claimed in claim 15 wherein the said central target element comprises a red target element.

17. Apparatus as claimed in claim 12 and arranged to present the said image for a predetermined period.

18. Apparatus as claimed in claim 17, wherein the period is arranged such that the said predetermined period is in the region of three seconds.

19. Apparatus as claimed in claim 12 and including means for presenting said image to each eye of a user.

20. Apparatus as claimed in claim 19, and arranged to repeatedly present said image to each eye for in the region of six times.

21. Apparatus as claimed in claim 12, including at least one electronic display screen for presenting the said image comprising a circle.

22. An apparatus as claimed in claim 12 and arranged for use in accordance with a method as claimed in claim 1.

23. An ophthalmic diagnostic apparatus as claimed in claim 12 and comprising: display means arranged to be worn in proximity to a user's eyes and arranged for displaying images relating to at least one visual function test for a user; a first user interface for providing instructions to a user relating to said at least one visual function test and a second user interface for receiving responses from said user in relation to said at least one visual function test; storage means for storing data relating to said at least one visual function test; control means for transferring said data relating to said at least one visual function test to said display means and said first user interface; and output means for outputting a signal responsive to the user responses so as to assist with ophthalmic diagnosis.

24. An apparatus according to claim 23, further comprising a processor for processing data relating to user responses to the at least one visual function test received via the second user interface and for forwarding processed data to said output means for output as said signal responsive to the user responses.

25. An apparatus according to claim 23, wherein said output means includes transmission means for transmitting said signal responsive to the user responses over a communication network.

26. An apparatus according to claim 25, wherein said signal responsive to the user responses is transmitted to a remote location.

27. An apparatus according to claim 26, wherein said communication network includes an intermediate transceiver element between said transmission means and said remote location, and wherein, upon reception of said signal responsive to the user responses, said intermediate transceiver is arranged to forward signal responsive to the user responses to said remote location.

28. An apparatus according to claim 25, wherein transmission of said signal responsive to the user responses over said communication network comprises wireless transmission.

29. An apparatus according to claim 26, wherein said control means further comprises a receiving means for receiving data transmitted over said communication network from a remote location.

30. An apparatus according to claim 1, wherein said output means is arranged to output said signal responsive to the user responses directly to said user.

31. An apparatus according to claim 1, wherein said control means is further arranged to forward data relating to said signal responsive to the user responses for storage in said storage means.

32. An apparatus according to claim 1, wherein said first user interface comprises visual means incorporated in said display means.

33. An apparatus according to claim 32, and arranged such that user instructions are presented to a user through said visual means.

34. An apparatus according to claim 1, wherein said display means is arranged to display three-dimensional images.

35. An apparatus according to claim 34, wherein said three-dimensional images are formed from a pair of two-dimensional images.

36. An apparatus according to claim 35, wherein a first two-dimensional image of said pair of two-dimensional images is displayed on a first screen of said display means and a second two-dimensional image of said pair of two-dimensional images is displayed on a second screen of said display means, where said first screen is arranged for location in proximity to one eye of the user and a second screen is arranged for location in proximity to the other eye of the user.

37. An apparatus according to claim 36, wherein said first image and said second image represent two perspectives of the same image to mimic the perspectives that both eyes of a user naturally receive in binocular vision.

38. An apparatus according to claim 1, wherein said first user interface comprises an audio signal transducer.

39. An apparatus according to claim 38, wherein said second user interface comprises the, or a further, audio signal transducer.

40. An apparatus according to claim 38, and arranged such that user instructions are presented to a user through an earpiece.

41. An apparatus according to claim 1, wherein said second user interface includes a microphone.

42. An apparatus according to claim 41, wherein said user responses are audio responses recorded via said microphone.

43. An apparatus according to claim 42, wherein said control means is configured to operate a voice recognition process for capturing and processing said audio responses of a user.

44. An apparatus according to claim 1, wherein said second user interface includes tactile elements.

45. An apparatus according to claim 44, wherein said tactile elements comprise user operable switches.

46. An apparatus according to claim 1, and arranged such that said at least one visual function test comprises a visual acuity test.

47. An apparatus according to claim 1, and arranged such that said at least one visual function test comprises a test for glaucoma.

48. An apparatus according to claim 1, and arranged such that said at least one visual function test comprises a test for monitoring distortion of vision.

49. An apparatus according to claim 1, and arranged such that said at least one visual function test comprises a colour vision test.

50. An apparatus according to claim 1, and arranged such that said at least one visual function test comprises a test for monitoring contrast sensitivity.

51. An apparatus according to claim 1, and arranged such that said at least one visual function test comprises a test for detecting changes in a retina.

52. An apparatus according to claim 1, further comprising an integral power source.

53. An apparatus according to claim 52, wherein said integral power source comprises a rechargeable battery.

54. An apparatus according to claim 1, wherein said apparatus is arranged in a headset.

55. An apparatus according to claim 1, wherein said apparatus comprises goggles.

56. An apparatus according to claim 55, wherein said display means is located in a lens portion of said goggles.

57. An apparatus according to claim 55, wherein said first user interface is located in a lens portion of said goggles.

58. An apparatus according to claim 55, wherein said first user interface is located in an earpiece portion of said goggles.

59. An apparatus according to claim 54, wherein said second user interface is arranged for location in proximity to a mouth of a user.

60. An apparatus according to claim 54, wherein said headset comprises spectacles.

61. An apparatus according to claim 1, wherein said display means comprises a liquid crystal display.

62. An apparatus according to claim 23, wherein said display means comprises an organic light emitting diode display.

63. A docking station for receiving the apparatus of claim 23, wherein said docking station comprises a protective case comprising a main housing for receiving the said ophthalmic diagnostic apparatus.

64. A docking station according to claim 63, further comprising a lid portion serving to enclose said ophthalmic diagnostic apparatus in said main housing when in a closed position and to allow access to said ophthalmic diagnostic apparatus when in an open position.

65. A docking station according to claim 63, further comprising a power supply cable to provide electrical power to said docking station and a means to electrically couple said ophthalmic diagnostic apparatus to said power supply when located within said docking station so as to recharge said rechargeable battery.

66. A docking station according to claim 63, when directly or indirectly dependent upon claim 5, wherein said intermediate transceiver element is located within said docking station.

67. An ophthalmic diagnostic system comprising: an ophthalmic diagnostic apparatus according to claim 23; a communication network; and a remote server, wherein a signal responsive to the user responses entered via said second user interface is transmitted from said ophthalmic diagnostic apparatus to said remote server via said communication network.

68. An ophthalmic diagnostic system according to claim 66, wherein data relating to corrective action proposed by an ophthalmic specialist based upon data received via said signal responsive to the user responses is transmitted from said remote server to said ophthalmic diagnostic apparatus via said communication network.

69. An ophthalmic diagnostic system according to claim 67, further comprising the docking station according to claim 63.

70. A computer program product including instructions such that, when loaded onto a computer, serve to provide for a method as defined in claim 1.

71. A computer program including a computer program product as defined in claim 70.

72. A record medium having a computer program as defined in claim 71 recorded thereon.
